(19) United States
(12) Patent Application Publication
(10) Pub. No.: US 2024/0104350 A1
(43) Pub. Date: Mar. 28, 2024

(54) MACHINE LEARNING-BASED INVARIANT DATA REPRESENTATION

(71) Applicant: RAMOT AT TEL-AVIV UNIVERSITY LTD., Tel Aviv (IL)

(72) Inventor: Ran Gilad BACHRACH, Tel Aviv (IL)

(21) Appl. No.: 18/273,342

(22) PCT Filed: Jan. 20, 2022
(86) PCT No.: PCT/IL2022/050085

§ 371 Date: Jul. 20, 2023

Related U.S. Application Data
(60) Provisional application No. 63/139,885, filed on Jan. 21, 2021.

(51) Int. Cl. G06N 3/045

(52) U.S. Cl. CPC G06N 3/045

(57) ABSTRACT

A system and method for predicting a condition of a subject may include one or more autoencoder modules, trained to: receive at least one content data element pertaining to the subject from one or more data sources of a plurality of data sources; and generate a source-invariant representation of the at least one content data element in a latent space of the one or more autoencoders. One or more machine-learning (ML) based classification models may receive the source-invariant representation of the at least one content data element, and produce therefrom a prediction data element, which may represent a predicted condition of the subject.

### CROSS REFERENCE TO RELATED APPLICATIONS

This application is a PCT patent application which claims the benefit of priority under 35 U.S.C. § 119(e) of U.S. Provisional Patent Application No. 62/594,550, filed Jan. 21, 2021, entitled “MACHINE LEARNING-BASED INVARIANT DATA REPRESENTATION”. The contents of the above applications are all incorporated by reference as if fully set forth herein in their entirety.

### FIELD OF THE INVENTION

The present invention generally relates to the field of machine learning based diagnostics. More specifically, the present invention relates to methods and systems for predicting a condition of a subject (e.g., a human subject or patient), based on invariant data representation.

### BACKGROUND

Data generated while people browse the Internet, especially when using Internet search engines, has been shown to reflect the experiences of people in the physical world. For example, many Internet users turn to search engines when they have a medical concern. For this reason, search queries and other online content have been used to track infectious diseases such as influenza, answer questions on the relationship between diet and chronic pain, and to identify precursors to disease.

However, harnessing the potential value of these data sources requires addressing their unique properties. Because each user may use a different set of Internet services, extracting useful standardized datasets requires handling different combinations of data sources. For example, the large number of different social networks, search engines, and instant messaging services creates a growing number of possible service combinations used by each individual user.

Another source of complexity is changing usage patterns over time, as different platforms fall in and out of favor with users. For example, in 2006, Facebook accounted for only a 5% share of social networking searches on Google, a number which grew to 90% in 2011, and declined to 71% by September 2020. Anther platform, MySpace, declined in search share from 90% in 2006 to 0% in 2014. Aggregating and standardizing data from multiple online sources must also account for a changing mix of sources over time, as well as changes in data structures and formats within particular platforms over time.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

There is provided, in an embodiment, a system that may include at least one hardware processor; and a non-transitory computer-readable storage medium having stored thereon program instructions, the program instructions executable by the at least one hardware processor to perform operations of a neural network, the neural network may include: one or more autoencoder neural networks, each configured to receive, as input, a set of content data elements (CDEs), wherein each of the CDEs is acquired from one of a plurality of Internet-based systems, and generate a common invariant representation of the input in a latent space, and an adversarial neural network configured to generate a set of candidate data instances in the latent space using a generative model, and discriminate between the representation and the candidate data instances using a discriminative model, wherein the neural network is trained in an unsupervised manner, and wherein the trained neural network is configured to generate a final representation of the input.

There is also provided, in an embodiment, a method that may include providing a neural network, the neural network may include: one or more autoencoder neural networks, and an adversarial neural network that may include a generative model and a discriminative model; at a training stage, training: (i) each of the one or more autoencoder neural networks on a training set that may include a set of CDEs acquired from one of a plurality of Internet-based systems, to generate a common invariant representation of the CDEs in a latent space; and (ii) the adversarial neural network to generate a set of candidate data instances in the latent space using the generative model, and discriminate between the representation and the candidate data instances using the discriminative model, wherein the training is unsupervised, and wherein the trained neural network is configured to generate a final representation of the set of CDEs.

There is further provided, in an embodiment, a computer program product that may include a non-transitory computer-readable storage medium having program instructions embodied therewith, the program instructions executable by at least one hardware processor to perform operations of a neural network, the neural network may include: one or more autoencoder neural networks, each configured to receive, as input, a set of CDEs, wherein each of the CDEs is acquired from one of a plurality of Internet-based systems, and generate a common invariant representation of the input in a latent space; and an adversarial neural network configured to generate a set of candidate data instances in the latent space using a generative model, and discriminate between the representation and the candidate data instances using a discriminative model, wherein the neural network is trained in an unsupervised manner, and wherein the trained neural network is configured to generate a final representation of the input.

In some embodiments, each of the CDEs is generated by a user using a specific Internet-based system.

In some embodiments, each of the CDEs is one of: an Internet search query; a posting to a social network; an email; a text message; and a transcription of a voice command.

In some embodiments, each of the CDEs may include one or more of: textual data, image data, video data, audio data, vocal data, and user-selection data.

In some embodiments, the plurality of specified internet-based system is one of: an internet search engine, an email services, an e-commerce website, a messaging application, a social network, and a virtual assistant device or software agent based on voice interaction.

In some embodiments, the discriminative model is configured to classify each of the candidate data instances as associated with one of the plurality of internet-based services.

Embodiments of the invention may include a system for predicting a condition of a subject.

Embodiments of the system may include one or more autoencoder modules, trained to receive at least one content data element pertaining to the subject from one or more data sources of a plurality of data sources; and generate a feature vector in a latent space of the one or more autoencoders. The feature vector may include a source-invariant representation of the at least one content data element.

Embodiments of the system may further include one or more machine-learning (ML) based classification models (also referred to herein as predictive models), trained to receive the source-invariant representation of the at least one content data element; and produce a prediction data element, representing a predicted condition of the subject, based on the source-invariant representation of said at least one content data element.

Additionally, or alternatively, embodiments of the system may include at least one adversarial neural network (NN) configured to predict, based on the source-invariant representation of the at least one content data element, an identification of an origin data source from which the at least one content data element originated.

For example, the identification of origin data source may include a data element (e.g., a name, a type, an identification number, etc.) that may indicate a specific type, subtype and/or platform, from which the at least one content data element was received, as elaborated herein (e.g., in relation to FIG. 2B).

Additionally, or alternatively, embodiments of the system may include at least one first training module configured to, during a training stage (e.g., an autoencoder training stage): receive a plurality of training content data elements from a plurality of data sources; and train the one or more autoencoder modules, based on the plurality of training content data elements, to generate the source-invariant representation such that the adversarial NN would fail in predicting the identification of origin data sources of one or more content data elements of the plurality of training content data elements.

According to some embodiments, the at least one first training module may be further configured to (e.g., during the autoencoder training stage): receive a plurality of annotation data elements, corresponding to the plurality of training content data elements; receive, from the one or more classification models, a plurality of prediction data elements, corresponding to the plurality of training content data elements; and train the one or more autoencoder modules further based on the prediction data elements and annotation data elements.

According to some embodiments, the plurality of annotation data elements may represent ground-truth information pertaining to a condition of corresponding subjects. The at least one first training module may be configured to train the one or more autoencoder modules to generate the source-invariant representation, such that the classification models correctly predict the conditions of relevant subjects, as represented by the annotation data elements.

Additionally, or alternatively, embodiments of the system may include one or more second training modules, corresponding to the respective one or more classification models. The one or more second training modules may be configured, during a training stage (e.g., during a classifier training stage) to: receive a plurality of source-invariant representations of a respective plurality of training content data elements; receive a plurality of annotation data elements, corresponding to the plurality of training content data elements; and train the one or more classification models to produce the prediction data elements, based on the plurality of source-invariant representations, using the annotation data elements as supervisory data.

It may be appreciated that the classifier model (or prediction model) training stage may be concurrent with the autoencoder training stage. Additionally, or alternatively, the classifier training stage may precede the autoencoder training stage. Additionally, or alternatively, the autoencoder training stage may precede the classifier training stage. Additionally, or alternatively, the classifier training stage and the autoencoder training stage may occur intermittently over time.

Embodiments of the invention may include method of predicting a condition of a subject by at least one processor.

Embodiments of the method may include receiving at least one content data element pertaining to the subject from one or more data sources of a plurality of data sources; applying one or more autoencoder models on the received at least one content data element, to generate a feature vector in a latent space of the one or more autoencoders and applying one or more ML-based classification models one the source-invariant representation of the at least one content data element, to produce a prediction data element. The feature vector may include a source-invariant representation of said at least one content data element, and the prediction data element may represent a predicted condition of the subject, such as a mental or cognitive condition (e.g., onset of a stroke or Alzheimer's disease) or a medical condition, such as onset of a degenerative disease.

Embodiments of the method may include, during an autoencoder training stage: receiving a plurality of training content data elements from a plurality of data sources; applying at least one adversarial NN on the source-invariant representation of the at least one content data element, to produce an identification of an origin data source, from which the at least one content data element was received; and training the one or more autoencoder modules, based on the plurality of training content data elements, to generate the source-invariant representation. The source-invariant representation may be generated such that the at least one adversarial NN would fail in predicting the identification of origin data sources of one or more content data elements of the plurality of training content data elements.

According to some embodiments, the at least one processor may (e.g., during the autoencoder training stage): receive a plurality of annotation data elements, corresponding to the plurality of training content data elements; receive, from the one or more classification models, a plurality of prediction data elements, corresponding to the plurality of training content data elements; and train the one or more autoencoder modules, further based on the prediction data elements and annotation data elements.

Additionally, or alternatively, the plurality of annotation data elements represent ground-truth information pertaining to a condition of corresponding subjects. The at least one processor may train the one or more autoencoder modules to generate the source-invariant representation, such that the classification models correctly predict the conditions of relevant subjects, as represented by the annotation data elements.

According to some embodiments, the at least one processor may (e.g., during the classifier training stage): receive a plurality of source-invariant representations of a respective plurality of training content data elements; receive a plurality of annotation data elements, corresponding to the plurality of training content data elements; and train the one or more classification models to produce the prediction data elements, based on the plurality of source-invariant representations, using the annotation data elements as supervisory data.

Additionally, or alternatively, embodiments of the method may include receiving a definition of a hierarchical categorization data structure, representing a plurality of hierarchical levels of the received content data elements. The at least one processor may apply the one or more autoencoder models on at least one content data element by generating a feature vector that includes a plurality of source-invariant representations of said at least one content data element, where each source-invariant representation of the feature vector corresponds to a respective hierarchical level.

According to some embodiments, the at least one processor may (e.g., during an autoencoder training stage) be configured to: receive a plurality of training content data elements from a plurality of data sources; and train the one or more autoencoder modules, based on the plurality of training content data elements, to generate said feature vector, while applying a predetermined weight to each source-invariant representations of the feature vector. The predetermined weight may be determined according to the hierarchical level of the respective source-invariant representation.

According to some embodiments, for each pair of source-invariant representations, said pair may include a first source-invariant representation corresponding to a first hierarchical level, and a second source-invariant representation corresponding to a second, higher hierarchical level. In such embodiments, the weight of the second source-invariant representation may be higher than the weight of the first source-invariant representation, to reflect the difference in hierarchical levels.

Additionally, or alternatively, the at least one processor may be configured (e.g., during a classifier training stage) to receive a plurality of feature vectors, corresponding to a respective plurality of training content data elements; receive a plurality of annotation data elements, corresponding to the plurality of training content data elements; and train the one or more classification models to produce the prediction data elements, based on the plurality of source-invariant representations, while (a) using the annotation data elements as supervisory data, and (b) applying a predetermined weight to each source-invariant representations of the feature vector. Said weight may be determined according to the hierarchical level of the respective source-invariant representation.

According to some embodiments, the at least one content data element may include a textual or audible data source, such as an Internet search query, a posting to a social network by the subject, an email pertaining to the subject; a text message pertaining to the subject, a transcription of a voice command pertaining to the subject, text included in a medical record pertaining to the subject, and the like.

Additionally, or alternatively, the at least one content data element may include an online data source, such as online user-selections performed by the subject, online images pertaining to the subject, online videos pertaining to the subject, and online audio or vocal data elements pertaining to the subject, and the like.

Additionally, or alternatively, the at least one content data element may include an image data source, such as an image of the subject, a video of the subject, a Magnetic Resonance Imaging (MRI) scan of the subject, a Computed Tomography (CT) scan of the subject, images obtained from an Ultrasound (US) scan of the subject, and the like.

Additionally, or alternatively, the at least one content data element may be a proteomic data element, a genomic data element, and the like.

Embodiment of the invention may include a system for predicting a condition of a subject. Embodiment of the system may include: a non-transitory memory device, wherein modules of instruction code may be stored, and at least one processor associated with the memory device, and configured to execute the modules of instruction code. Upon execution of said modules of instruction code, the at least one processor may be configured to: receive at least one content data element pertaining to the subject from one or more data sources of a plurality of data sources; apply one or more autoencoder modules on the at least one content data element to generate a feature vector in a latent space of the one or more autoencoders, said feature vector including a source-invariant representation of said at least one content data element; and apply one or more ML based classification models on the source-invariant representation of the at least one content data element, to produce a prediction data element, representing a predicted condition of the subject.

In addition to the exemplary aspects and embodiments described above, further aspects and embodiments will become apparent by reference to the figures and by study of the following detailed description.

### BRIEF DESCRIPTION OF THE FIGURES

The present invention will be understood and appreciated more comprehensively from the following detailed description taken in conjunction with the appended drawings in which:

FIG. 1 is a block diagram, depicting a computing device which may be included in a system for predicting a condition of a subject, according to some embodiments;

FIG. 2A is a schematic drawing, illustrating collection of content data elements (CDEs), according to some embodiments of the present invention;

FIG. 2B which is a schematic diagram, illustrating an example of a hierarchical categorization of CDEs 21, which may be utilized by embodiments of the present invention to classify a condition (e.g., a medical condition) of a human subject;

FIG. 3 is a schematic flow diagram, depicting an overview of a process for training a machine learning model for early prediction of a cerebrovascular disease, such as a stroke, according to some embodiments of the present invention;

FIG. 4A is a schematic block diagram depicting a system for classifying or predicting a condition of a subject, based on invariant representation of content data elements, according to some embodiments of the invention;

FIG. 4B is a schematic block diagram depicting additional or alternative aspects of the system for classifying or predicting a condition of a subject, according to some embodiments of the invention;

FIG. 4C is a schematic block diagram depicting additional or alternative aspects of the system for classifying or predicting a condition of a subject, according to some embodiments of the invention; and

FIG. 5 is a schematic flow diagram depicting a method of classifying or predicting a condition of a subject, based on invariant representation of content data elements, according to some embodiments of the invention.

### DETAILED DESCRIPTION

Disclosed are a method, system, and computer program product for generating standardized invariant latent space data representations of content generated by users using multiple Internet services and platforms.

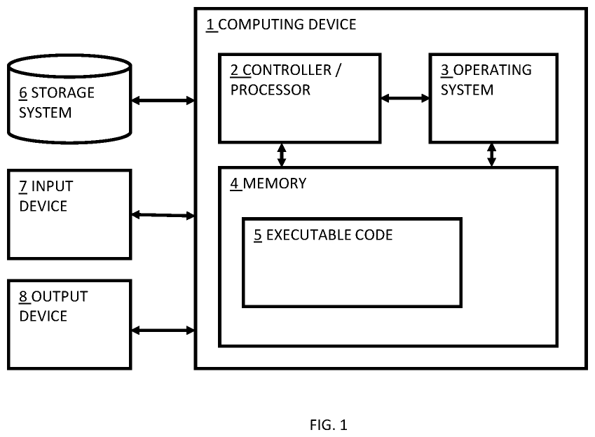

Reference is now made to FIG. 1, which is a block diagram depicting a computing device, which may be included within an embodiment of a system for predicting a condition of a subject (e.g., a human subject or patient), based on invariant data representation, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may predict a condition of a subject, as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to a condition of a subject may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse, and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

In some embodiments, the present invention provides for acquiring and aggregating CDEs generated by users over multiple Internet services and platforms, e.g., in the course of regular usage of online and related sources.

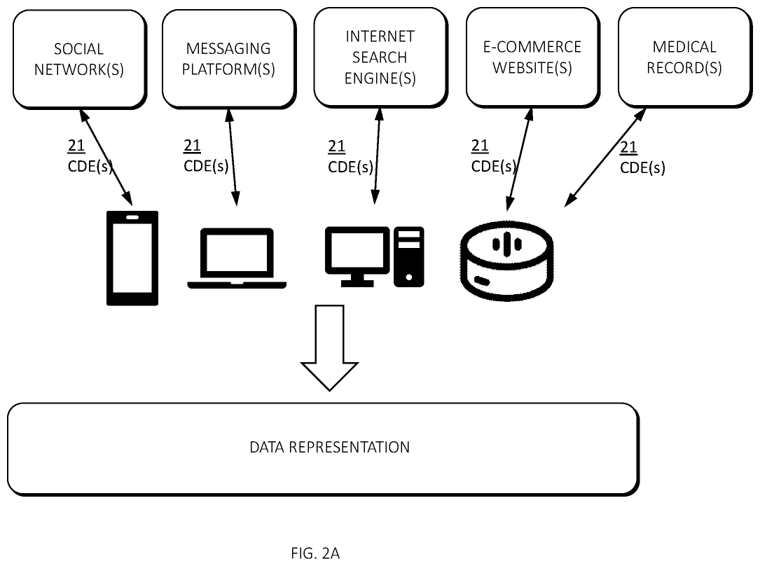

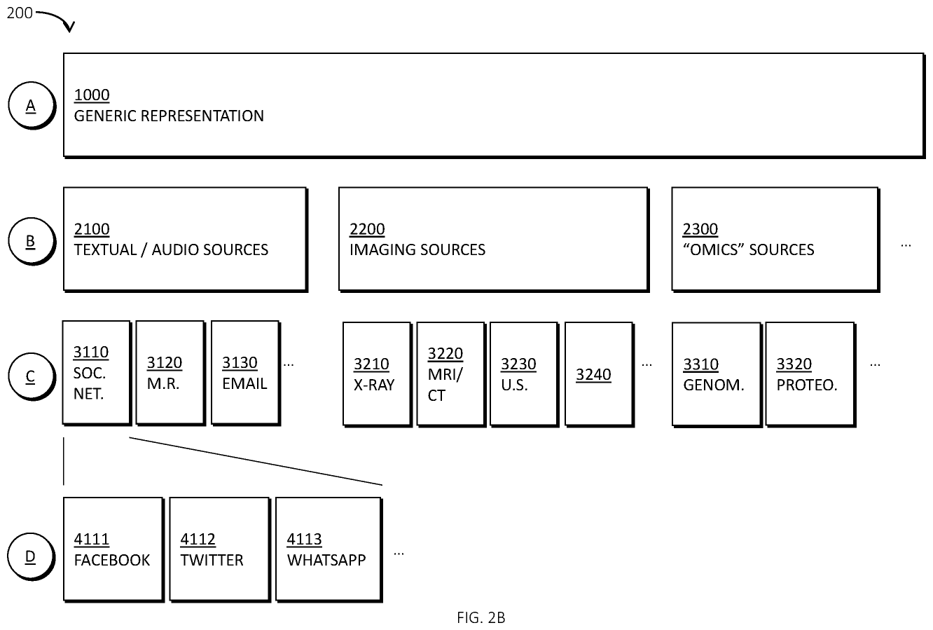

Reference is now made to FIG. 2A which is a schematic block diagram, illustrating collection of CDEs 21, also referred to herein as “content segments” according to some embodiments of the present invention. As shown in FIG. 2A, embodiments of the invention may acquire content segments or CDEs 21 from one of a plurality of Internet-based systems, related to a user's online activity. This acquisition of content segments or CDEs 21 may be based, at least in part, on monitoring computing devices used by the user to engage in online activity, e.g., a personal computer, a laptop computer, a mobile device, a tablet, a smartphone, a smart watch, a voice-operated personal assistant device or software agent, and the like.

In some embodiments, the online activity may include a user's regular, day-to-day online activity, and may refer to a plurality of activity types using a variety of Internet service provider platforms and services, e.g., online searches, Internet commerce activity, social media posting, and/or instant messaging, over a variety of sources, e.g., search engines (Google, Bing), e-commerce websites (Amazon), messaging applications (WhatsApp, Snapchat, SMS messages, MMS messages), social media platforms (Facebook, Twitter, Instagram, LinkedIn), virtual assistant devices or software agents based on voice interaction (Amazon Alexa, Apple's Siri, Microsoft Cortana), etc.

In some embodiments, online CDEs may be acquired from any one or more computing devices used by a user to engage in online activity, e.g., a personal computer, a laptop computer, a mobile device, a tablet, a smartphone, a smart watch, a voice-operated personal assistant device or software agent, and the like.

It is noted that online activity data and content generated by Internet users and posted and/or exchanged online using an Internet service or platform may not always be readily accessible to users. However, more recently, privacy and related legislation in many jurisdictions worldwide, e.g., the European General Data Protection Regulation (GDPR), the California Consumer Privacy Act (CCPA), encodes a user's right to access and transfer data which a service provider holds with respect to the user. Thus, in many jurisdictions, users have the ability to gain access to data that service providers collect about them, in a machine-readable format.

Additionally, or alternatively, one or more CDEs 21 may include for example textual data representing Internet search queries generated by users, data representing social media posts and/or comments made by users, data representing voice commands expressed by the users, text messages exchanged by users with other individuals and/or groups over a communication network (e.g., the Internet), and the like.

Additionally, or alternatively, one or more CDEs 21 may be, or may include for example Internet Service Providers Data (ISPD), e.g., data collected by different service providers such as search engines, social media, e-commerce sites, instant messaging services, and the like.

Additionally, or alternatively, one or more CDEs 21 may be, or may include for example textual or audible data sources, such as an Internet search query; a posting to a social network by the subject; an email pertaining to the subject; a text message pertaining to the subject; a transcription of a voice command pertaining to the subject; and text included in a medical record pertaining to the subject.

In some embodiments, one or more CDEs 21 may be acquired continuously or repeatedly over a specified period of time. In some embodiments, the acquired and aggregated CDEs 21 may be textual, and may be typed or input by a user via a user interface.

For example, CDEs 21 may be input or introduced by a user when entering an online search query via a web browser. In another example, CDEs 21 may be input by a user when exchanging text messages (e.g., via an online chat). In another example, CDEs 21 may be input by a user when posting a message via a messaging service (e.g., a short messaging service (SMS)), over a communication network (e.g., a cellular communication network, the Internet, and the like).

In some embodiments, CDEs may represent short text data elements such as a content of a search query. Additionally, or alternatively, data of the CDEs may represent longer textual interactions such as posts on a social network or discussion forums.

In some embodiments, CDEs may be, or may include or represent an image, a video, and/or any other visual data.

Additionally, or alternatively, one or more CDEs may be, or may include for example image data sources such as an image of the subject, a video of the subject, a Magnetic Resonance Imaging (MRI) scan of the subject, a Computed Tomography (CT) scan of the subject, and images obtained from an Ultrasound (US) scan of the subject.

Additionally, or alternatively, an acquired CDE 21 may be, or may include or represent audio-based content and/or voice-based content that may, for example be obtained by recording voice commands spoken by a user.

Additionally, or alternatively, the acquired CDE 21 may be or may include a transcribed, textual version or form of recorded voice content. In other words, the audio-based CDE may be transcribed into textual representation using any suitable voice recognition technique, and later used by embodiments of the invention in the textual form.

Additionally, or alternatively, one or more CDEs may be, or may include for example proteomic data elements, representing proteomic information pertaining to the subject and genomic data elements, representing genomic information pertaining to the subject.

In some embodiments, the acquired CDE may include indication of user selection on a web page, as detected by a web browser. For example, the acquired CDE may include a representation of selection (e.g., clicking on) hyperlinks in a web page, selection of menu options in the web page, selection of purchase options in the web page, and the like.

Additionally, or alternatively, one or more CDEs may be, or may include for example online data sources, such as online user-selections performed by the subject, online images pertaining to the subject, online videos pertaining to the subject, and online audio or vocal data elements pertaining to the subject.

Reference is now made to FIG. 2B which is a schematic diagram, illustrating an example of a hierarchical categorization of CDEs 21, which may be utilized by embodiments of the present invention to classify a condition (e.g., a medical condition) of a user.

As shown in FIG. 2B, embodiments of the invention may receive (e.g., from input device 7 of FIG. 1) a hierarchical definition or categorization 200 of CDEs 21. Hierarchical categorization 200 may, for example be organized according to data types, data subtypes and/or data origins. Embodiments of the invention may apply one or more machine-learning (ML) based models on the obtained CDEs 21, to classify or predict a condition (e.g., a medical condition) of a human subject, in a manner that is oblivious of CDE 21 types, subtypes and/or origins, as defined by hierarchical categorization 200.

In other words, embodiments of the invention may be configured to predict or identify a condition of a human subject, based on CDEs 21, in a manner that is independent of types, subtypes and/or origins of the obtained CDEs 21.

For example, a user's medical condition may include a condition of a progressing stroke. Indications of this medical condition may be manifested in a plurality of levels, and may be obtained from a plurality of sources or origins, each having a specific type. In the example of FIG. 2B, the condition of a progressing stroke may be manifested in a plurality of CDE source types, such as textual and/or audio CDE 21 source types 2100, imaging CDE source types 2200, and “Omics” (e.g., genomics, proteomics) CDE source types 2300. Other CDE source types may also be possible. The high-level unification of all CDE source types is denoted in FIG. 2B as the “Generic representation” CDE source type 1000.

The categories of CDEs 21 may be referred to as hierarchical, in a sense that each CDE source type (e.g., 1000, 2100, 2200, 2300) may include one or more CDE source subtypes, in which the condition of the human subject may be manifested.

For example, the textual and/or audio CDE source types 2100 may include audible and/or textual data elements in a social network platform 3110 (e.g., text posted by the human subject in “Facebook”, or sent over a chat platform such as “WhatsApp”), structured, or unstructured textual data that is included in medical records 3120 (e.g., written by a physician or a caretaker), textual data that is included in email messages 3130, received by, or sent from a computing device or an email account associated with the relevant human subject, and the like.

In another example, imaging CDE source types 2200 may include data obtained from medical imaging systems, including for example X-ray images 3210, images obtained from Magnetic Resonance Imaging (MRI) systems or Computed Tomography (CT) systems 3220, images obtained from Ultrasound (US) systems 3230, and the like.

Additionally, imaging CDE source types 2200 may include images or videos of the human subject 3240 (e.g., on a computing device pertaining to the human subject), and/or images or videos posted by the human subject on (e.g., on a cloud-based computing device or server).

In another example, “Omics” CDE source types 2300 may include data that is obtained by a DNA genomic test of the human subject 3310, proteomic test of the human subject 3320, and the like.

Additionally, each subtype of hierarchical categorization 200 may further include, or may be further divided to additional, fine-grain subtypes or origins. For example, a category 3110 of social networking text CDEs 21 may further include specific origins or platforms of social media, including for example Facebook-originated CDEs 4111, Twitter-originated CDEs 4112, WhatsApp-originated CDEs 4113, and the like.

In a similar example, further granularity may be obtained with other modalities: For example, there could be differences between MRI machines. Therefore, MRI source types 3220 may be elaborated in another layer, which may account for differences between specific machines (e.g., newer machines may have higher power and greater resolution).

It may be appreciated that manifestation of a condition of a human subject may be different in each of the CDE source types, as demonstrated by the hierarchical categorization 200 example of FIG. 2B. Additionally, the difference in manifestation may be perceived as larger among CDEs 21 that are more distantly related.

Pertaining to the example of a medical condition of a stroke, a manifestation of this condition in textual CDE sources 2100 may include properties of a subject's textual expression (e.g., whether text written by the subject involves spelling mistakes). Manifestation of a stroke in imaging CDE sources 2200 may include segmentation of images (e.g., MRI images) showing an area suspected as depicting a stroke. Manifestation of a stroke in “Omics” CDE sources 2300 may include a representation of expression of a stroke-related protein in a sample taken from the subject. These manifestations may be regarded as very different, as they may be expressed by different modalities, and represent different aspects of the subject's condition. As the granularity of CDE sources is increased (e.g., moving to lower layers of the hierarchical categorization 200), so are the nuances of manifestation of a stroke. For example, within the category of textual CDE sources 2100, a difference in manifestation of a stroke between (a) text that is included in a social media 3110 correspondence (e.g., a “tweet” on “twitter” 4112) and (b) text that is included in an email correspondence 3130 may be defined, or augmented by the nuances in the difference between the specific textual platforms. Such nuances may include, for example the style of correspondence (e.g., informal in a tweet, formal in an email), the length of correspondence (e.g., short messages in a tweet, in contrast to lengthy messages in emails), and the like.

In other words, the hierarchical categorization 200 may be regarded as a map of common denominators of manifestation of a human subject condition, where the higher levels of hierarchical categorization 200 (e.g., levels A, B) represent high-level, generic common denominators, and the lower levels of hierarchical categorization 200 (e.g., levels C, D) represent low-level, source-specific or platform-specific common denominators.

As elaborated herein, embodiments of the invention may be configured to extract source-independent or platform-independent, invariant representations 110B of CDEs 21, and utilize the invariant representations 110B to correctly predict the condition of the human subject, in a manner that is independent of the data source (the CDE 21 source). Additionally, embodiments of the invention may be configured to extract the invariant representation 110B of CDEs 21 according to the hierarchical categorization 200, so as to prefer an invariant representation 110B that is as hierarchically high as possible, as elaborated herein.

It may be appreciated that each user may use a different set of services and platforms. Therefore, extracting useful properties of the data, e.g., for use as training data to train predictive models, may require the capacity to handle the various combinations of data sources available for different users. Given that the popularity of each of these internet services changes over time, the set of particular sources may also change over time to include new and different sources. Furthermore, even for a single data source, any solution should account for potential changes in the data and its format due to product and engineering decisions of the service provider, as well as changes in usage patterns by users. Furthermore, even the specifics of the acquisition devices and protocols create differences that can affect trained models significantly.

Accordingly, in some embodiments, the present invention provides for acquiring, aggregating, and creating an invariant latent space representation of aggregated CDEs 21 generated by one or a plurality of users, as acquired from one or a plurality of online services and sources used regularly by each of the users. In some embodiments, each of the Internet sources used by any of the users may define a different data type, content, structure, and/or format. In some embodiments, each of the users may engage with a particular combination or set of sources, which may differ from combinations or sets of sources used by any other of the users. In some embodiments, the combination or set of sources used by any particular user may change and evolve over time, to include additional, fewer, other, and/or different one or more sources. For example, the data for any particular user may include a different mix of online sources, wherein such mix may vary over time in terms of a mix of sources and platforms used by the user.

In some embodiments, the present invention provides for aggregating the acquired CDEs 21 from the various sources, based on transforming the various types, structures, and/or formats of content into a normalized and/or standardized latent space representation. In some embodiments, the present invention provides for transforming online usage and activity content acquired from a diverse set of sources, into a unified latent space representation which is invariant to the data source, using machine learning and deep learning techniques.

In some embodiments, the present invention provides for an encoding machine learning model which generates a unified invariant representation of the multiple data sources. In some embodiments, the unified invariant representation may be invariant to user identity, such that the processed data may not be traced back to, linked with, and/or associated with any particular user. In some embodiments, the unified invariant representation may also be invariant to the data source, such that the processed data may not be traced back to, linked with, and/or associated with any particular Internet service type, Internet service provider, and/or Internet platform. This approach also reduces any privacy risks stemming from the use of data originating from Internet activity by individuals.

It may be appreciated that representation Embodiments of the invention may be combined with privacy preserving technologies to protect the privacy of data owners both during a training phase and during a testing time. Such privacy technologies include, but are not limited to, homomorphic encryption, secure multi-party computation, differential privacy, k-anonymity, obfuscation, PII removal, and the like.

In some embodiments, such acquired, aggregated, and standardized unified invariant latent space representation, may be used for constructing training datasets for training and inferring various machine learning prediction and classification models.

In some embodiments, CDEs 21 associated with the usage by users of different Internet services and platforms (e.g., search engines, social media, e-commerce sites, instant messaging services), may serve as a useful source in assessing cognition in users. Because these services are tightly integrated into the daily lives and routines of most individuals, and because these data reflect many cognitive and mental functions, it possesses the properties needed to enable prediction of states related to cognition.

Thus, the online usage and activities by the users may be indicative of everyday cognition in the users, because they typically require motor function to operate a keyboard and move a mouse; language processing to comprehend, select, retrieve, and compose textual expressions; and executive function to plan, inhibit, focus, and shift attention. Routine online use, such as querying of search engines, has been shown to correlate with standard cognitive tests and may thus be used as a continuous, unobtrusive, and cost-effective monitoring application.

Accordingly, the present invention will discuss most prominently uses of such datasets constructed according to embodiments of the present invention in conjunction with predictive machine learning models configured to predict various health-related states in users, wherein the health-related states may be correlated with impaired and/or declining cognition in the users and/or may be manifested in particular cognitive and/or mental symptoms. In some embodiments, such health-related states may include, e.g., cerebrovascular diseases, neurological diseases, psychopathologies, mental disorders, cognitive disorders, including, but not limited to: schizophrenia; neurocognitive disorders; bipolar and related disorders; anxiety disorders (generalized anxiety disorder, social anxiety disorder, panic disorder); stress-related disorders; dissociative disorders; somatic symptom disorders; eating disorders; disruptive disorders; depressive disorders; obsessive-compulsive disorders; personality disorders; and/or substance abuse related disorders.

However, aspects of the present invention may be used for constructing training datasets for training and inferencing various machine learning prediction and classification models configured to predict a wide range of diseases, medical and/or mental conditions, illnesses, syndromes, medical and/or mental disorders, personal health and/or other states, e.g., any acquired disease, chronic condition, congenital disorder, genetic or hereditary condition, etc.

Some aspects of the present invention provide for a method, system, and computer program product for predicting various health-related states in users, wherein the health-related states may be correlated with impaired and/or declining cognition in the users and/or may be manifested in particular cognitive symptoms.

In some embodiments, the present invention provides for training one or more predictive machine learning models on datasets generated according to the present invention, and representing online activity of a cohort of users. In some embodiments, with respect to each user in the cohort, the data may be collected periodically, over a defined period of time, over defined time windows, and/or continuously. In some embodiments, with respect to each user in the cohort, the data may represent a plurality of online activity types, e.g., online searches, Internet commerce activity, social media posting, and/or instant messaging, over a variety of Internet sources and platforms, e.g., search engines, e-commerce websites, messaging applications, social media platforms, and/or virtual assistant devices or software agents based on voice interaction. In some embodiments, with respect to each user in the cohort, the data sources may represent a different mix of activity types and/or data sources and platforms.

In some embodiments, a trained predictive machine learning model of the present invention may be applied to one or more target datasets, e.g., one or more representations of online activity by one or more target users, to predict one or more health and/or mental states in any of the target users. In some embodiments, online activity and engagement by each of the target users may be monitored continuously and/or periodically, to generate continuous target datasets that may include standardized, invariant latent space representation of the online activity by each of the users. In some embodiments, a predictive machine learning model of the present invention may be applied to the continuously-generated target datasets, to predict in real time one or more health and/or mental states in any of the target users.

In some embodiments, the present invention may provide for early prediction of a risk of a cerebrovascular event, e.g., an impending stroke, in a target user. In some embodiments, a predictive machine learning model of the present invention may be trained to predict a risk for an impending stroke within a following specified time period, e.g., within 5 days.

Accordingly, in some embodiments, the present invention provides for monitoring, in an ongoing manner, of users' online activity using aggregation of data from multiple sources, on the assumption that changes in the cognition of the user, e.g., in a period leading up to a cerebrovascular event, may be represented in the user's cognitive-related activity. In some embodiments, the present invention provides for aggregation of data representing users' cognition from multiple sources, on the assumption that changes in the cognition of the user, e.g., in a period leading up to a cerebrovascular event, may be represented in the user's cognitive-related activity. In some embodiments, the data received from a set of invariant representations may be used as input to a machine learning model trained to predict an impending stroke, as well as a prediction of time to the projected event, e.g., predict at time t the risk for stroke in the next, e.g., 5 days, using data acquired over, e.g., the preceding 90 days.

In some embodiments, the present invention provides for early prediction of an impending stroke, by detecting the presence of a covert cerebrovascular disease which often precedes stroke. Commonly applied risk assessment tools for stroke are based on the Framingham equation, which identified traditional cardiovascular (CV) risk factors, including hypertension, dyslipidemia, and diabetes, from a large community-based cohort of adults. The prevalent models for stroke prediction, such as those of the American College of Cardiology/American Heart Association (ACC/AHA) CVD risk algorithm and the Framingham Risk Score (FHS) offer a more general predictive target for a vascular “event” including stroke, myocardial infarction, death from coronary heart disease, congestive heart failure, incident angina, or intermittent claudication. Furthermore, they calculate the likelihood of event in the near decade, rather than able to predict an impending short-term cardiovascular threat.

An alternative approach for predicting stroke relies on identification of covert cerebrovascular disease which often precedes stroke. Covert cerebrovascular disease is associated with subtle cognitive and motor deficits and increased risk for stroke and further cognitive decline. One such covert disease is transient ischemic attacks (TIA), often regarded as mini-strokes, which are associated with a substantial increase in short-term risk of stroke (3-10% in the next 2 days, and 9-17% in the next 90 days). Currently, TIA and similar diseases can be diagnosed by either neuroimaging or in-person assessment using conventional cognitive screening tests. However, both methods are costly, have limited efficacy, and do not provide for continuous monitoring. Thus, most TIA cases go undiagnosed, and therefore, to date, it has not been harnessed into stroke prediction methods. However, because TIA creates at least a short term cognitive or mental disability, it may be possible to detect these temporal impairments using, e.g., machine learning techniques.

Accordingly, in some embodiments, the present invention provides for early prediction of an impending cerebrovascular event (such as a stroke), by detecting the presence and/or escalation of a covert cerebrovascular disease, based, at least in part, on continuous monitoring of cognitive activity of a user in performing regular day-to-day online activities, wherein these activities may be a source for assessment of everyday cognition in the user, because they typically require motor function to operate the keyboard and move the mouse; language processing to comprehend, select, retrieve, and generate appropriate words; and executive function to plan, inhibit, focus, and shift attention. Routine use, such as querying of search engines, has been shown to correlate with standard cognitive tests and may thus be used as a continuous, unobtrusive, and cost-effective monitoring application.

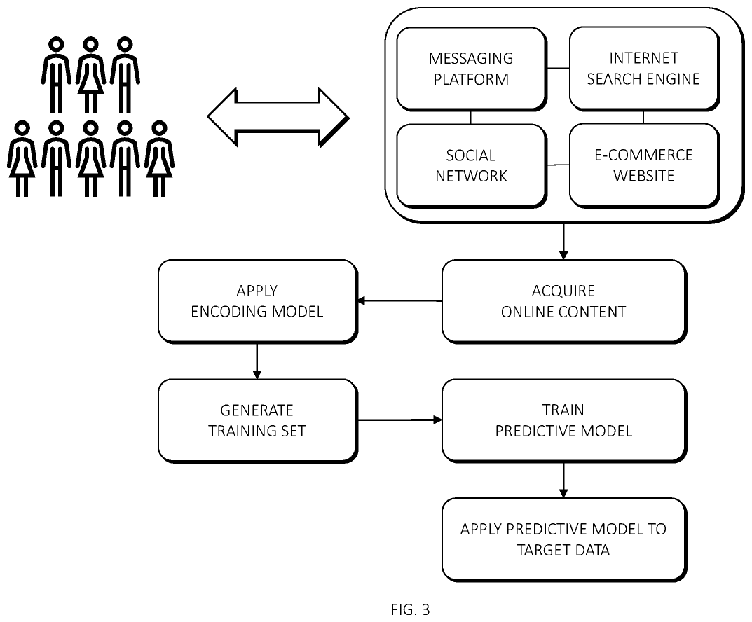

Reference is now made to FIG. 3 which is a schematic overview of the functional steps in a process for generating standardized invariant latent space data representations of CDEs 21 generated by users using multiple Internet services and platforms, according to some embodiments of the present invention.

As noted above, in some embodiments, the present invention provides for continuously acquiring CDE 21 generated by users in the course of regular usage of online and related sources, wherein such usage may be a source for assessment of everyday cognition in the user, because it typically requires motor function to operate a computing device; language processing to comprehend, select, retrieve, and generate appropriate words; and executive function to plan, inhibit, focus, and shift attention.

In some embodiments, CDEs 21 generated based on content from online usage may be indicative and/or associated with a wide range of diseases, medical and/or mental conditions, illnesses, syndromes, medical and/or mental disorders, personal health and/or other states, e.g., any acquired disease, chronic condition, congenital disorder, genetic or hereditary condition, etc.

In some embodiments, the present invention may provide for aggregating the acquired CDEs 21 from the various sources into a dataset representing content generated by one or more users in the course of engaging in online activities using textual and/or verbal interaction modalities.

In some embodiments, the data representation of the CDEs 21 may include a semantic analysis of the content, e.g., identifying and detecting concepts representing semantic meaning of specific expressions in the content, e.g., keywords entered in a search query, for example, personal references, mention of relevant symptoms, or mention of certain medications.

In some embodiments, the data representation may reflect sentiment analysis of the content, e.g., detected affective states of a user.

In some embodiments, the data representations may include features associated with the content, including one or more of:
Number of words in a CDE 21 (e.g., search query, social media post, text message);
time of day of generating a CDE 21;
a likelihood of generating a particular query string within a cohort of users;
number of words in a current CDE 21 that are not present in a user's other or previous CDEs 21;
changes in vocabulary used by a particular user;
number of spelling mistakes and use of automatic spelling correction;
elapsed time since a most recent generated content by a user;
number of CDEs 21 generated by a user per a specified time period (e.g., day, hour);
engagement with results of the generated content (e.g., search results of a search query), for example, time-to-click on a displayed result, time to reply to a message, time to comment on a viewed social media posting;
previous usage of a same CDE 21 (e.g., same search query) in the past.

In some embodiments, the acquired CDEs 21 may be aggregated and transformed into a normalized and/or standardized representation. In some embodiments, such representation may be generated by an encoding machine learning model which represents probability distributions over the aggregated CDEs 21.

As noted above, online usage and engagement patterns vary by user and over time, both in terms of the types of activities, as well as the specific services and platforms being used. Accordingly, using only a single data source (e.g., only Google searches or Facebook postings) will severely limit the ability to capitalize on many aspects on online activity which correlate with user cognition, because the usage of that single source may be limited or vary among different users, and usage patterns may shift such that this particular source may fall out of favor over time, wherein an alternate source may need to be sought.

Thus, embodiments of the present invention may include an improvement over currently available technology by generating a standardized data representation by that may aggregate data from a plurality of disparate sources. The ability to continuously acquire usable data over multiple changing sources may ensure the efficacy and sustainability of the present predictive model, in view of changing Internet usage patterns.

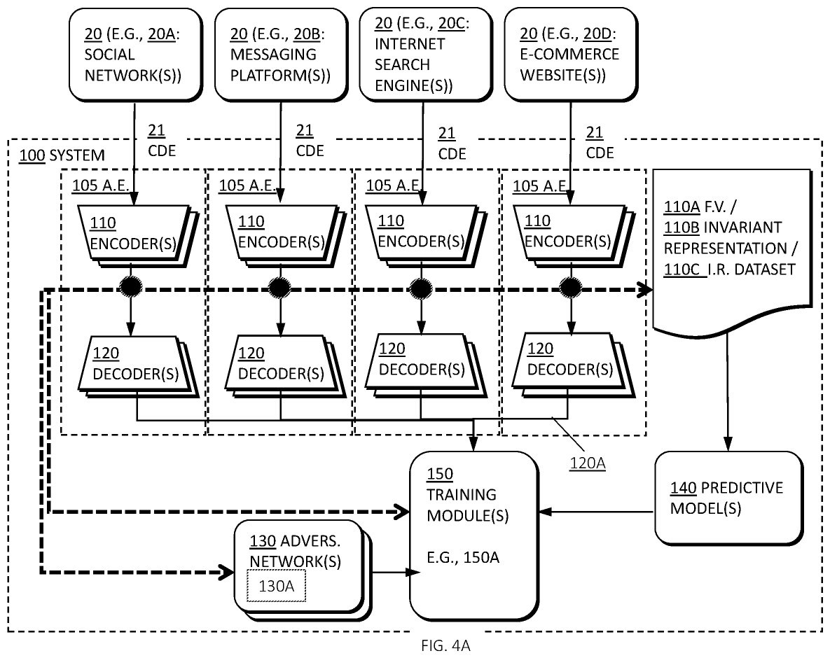

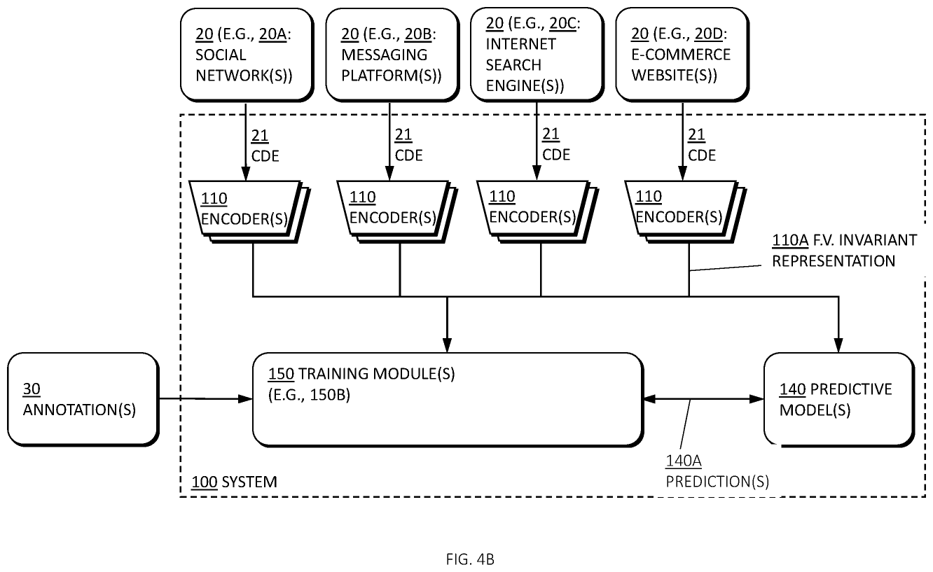

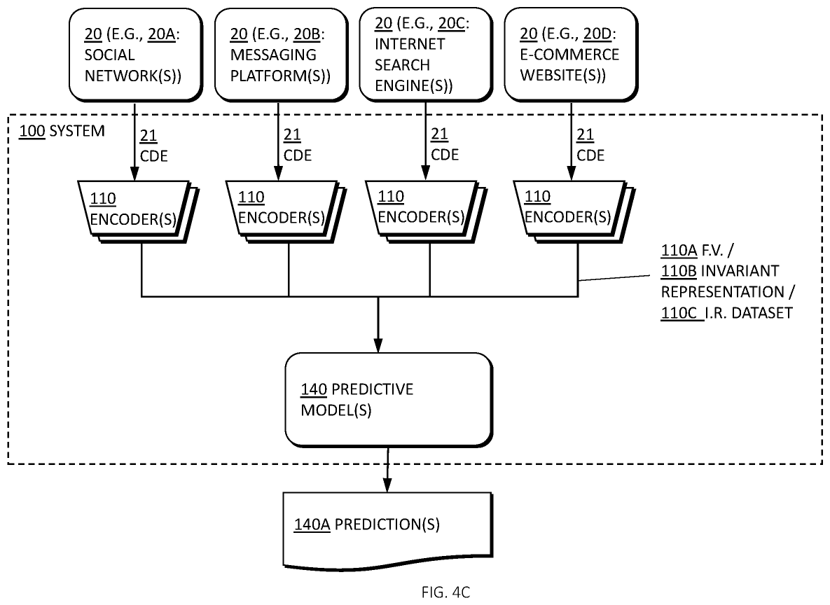

Reference is now made to FIG. 4A which is a schematic block diagram depicting a system 100 for classifying or predicting a condition of a subject, based on invariant representation of content data elements, according to some embodiments of the invention.

According to some embodiments of the invention, system 100 may be implemented as a software module, a hardware module, or any combination thereof. For example, system 100 may be, or may include a computing device such as element 1 of FIG. 1, and may be adapted to execute one or more modules of executable code (e.g., element 5 of FIG. 1) to predict a condition of a subject (e.g., a human subject or patient), as further described herein. As shown in FIG. 4A, arrows may represent flow of one or more data elements to and from system 100 and/or among modules or elements of system 100. Some arrows have been omitted in FIG. 4A for the purpose of clarity.

As shown in FIG. 4A, system 100 may include one or more machine-learning (ML) based autoencoder modules 105. As known in the art, an autoencoder may be a type of artificial neural network, used to produce an encoding or representation for a set of data, typically for dimensionality reduction, by training the network to ignore insignificant data. In some embodiments, each autoencoder module 105 may include one or more encoder 110 modules 110 and corresponding one or more decoder modules 120.

According to some embodiments, encoder 110 modules may receive at least one CDE 21 pertaining to a subject (e.g., a human subject) or patient from one or more data sources 20 of a plurality of data sources. In the example of FIG. 4A, the plurality of data sources 20 include social networks 20A, messaging platforms 20B, internet search engines and e-commerce websites.

Pertaining to the same example, corresponding CDEs 21 may include social network content, text messages, content of internet search queries, and data pertaining to e-commerce web pages.

According to some embodiments, encoder 110 modules may include a neural network architecture for performing operations of encoding content data elements 21 into a source-invariant representation 110B. In a complementary manner, decoder 120 modules 120 may include a neural network architecture for decoding the source-invariant representation 110B back to a restored version 120A of the input CDE 21.

It may be appreciated that an input CDE 21 may include one or more explicit, or implicit features that may associate the CDE 21 with a specific data origin. For example, a non-formal short text may characterize a textual CDE 21 as originating from a first data source or platform (e.g., a WhatsApp text message), whereas a formal, lengthy text may characterize a textual CDE 21 as originating from a different data source or platform (e.g., an email)

According to some embodiments, each encoder 110 module may be adapted to encode at least one input CDE 21 of a respective, specific CDE type or source, so as to generate a respective source-invariant representation 110B of the at least one (e.g., each) CDE 21 obtained from the one or more (e.g., each) data sources 20. Data element 110B may be referred to as “source-invariant” in a sense that it may be devoid of data that may allow reconstruction or prediction of a source or origin of the relevant CDE 21 by respective decoder modules 120.

In other words, encoder 110 may be configured to produce a source-invariant representation 110B of a received CDE 21 in a latent space of the autoencoder, such that a respective decoder 120 may not be able to predict, or classify predefined characteristics of the received CDE 21.

For example, encoder(s) 110 may be configured to produce source-invariant representation 110B so as to be invariant, or devoid of representation of an origin or source of CDE 21, causing decoder modules 120 to fail in ascertaining or predicting a source 20 or origin of CDE 21.

In another example, encoder(s) 110 may be configured to produce source-invariant representation 110B so as to be invariant, or devoid of relating to changes in the data collection process. In other words, encoder(s) 110 may generates a representation 110B that is capable of handling different available data sources and is invariant to changes in the data sources. Thus, encoding models 110 may significantly broaden the range of possible data sources.

As shown in FIG. 4A, different sources data sources 20 may be used as CDE 21 input into a set of autoencoder neural networks 105. In some embodiments, each autoencoder 105 may be unique, or dedicated to a specific data source 20. The dedicated encoders 110 of autoencoder 105 neural networks may be configured to convert the CDE 21 input data into a common invariant representation 110B data element.

In some embodiments, an encoding machine learning model of the present invention may be configured to learn a representation of a set of data instances, wherein a data input into the trained model may include a set input, e.g., a set of data streams acquired from a plurality of diverse sources. In some embodiments, an encoding machine 110 learning model of the present invention may be permutation invariant, wherein an output of the model does not change under any permutation of the elements in the input CDE 21 set of data sources. In some embodiments, an encoding machine 110 learning model of the present invention may be configured to process input sets of varying sizes.

In some embodiments, the present invention employs autoencoder neural networks which learn an efficient data coding in an unsupervised manner. The aim of the autoencoders is to learn a representation (encoding) for a set of data. Along with the encoders' 110 reduction side, a decoder 120 or reconstructing side is learnt, where the autoencoder 105 tries to generate from the reduced encoding a representation as close as possible to its original input CDE 21. The learned invariant representation 110B of the CDE 21 input may assume useful properties.

According to some embodiments, autoencoder model(s) 105 may include a “bottleneck” layer of one or more neural nodes. As known in the art, such a “bottleneck” layer may include a feature vector 110A representation of the input CDE 21 in a reduced dimension, commonly referred to as a “latent” dimension.

In some embodiments, invariant representation 110B may be taken from the values of the feature vector 110A (e.g., the one or more neural nodes of the “bottleneck” layer).

Additionally, or alternatively, autoencoder model(s) 105 may generate a feature vector 110A in a latent space of the one or more autoencoders. Feature vector 110A may be, or may include one or more source-invariant representation 110B of at least one input CDE 21.

Additionally, or alternatively, invariant representation 110B may be a data structure (e.g., a vector, an array, etc.) which includes a plurality of neural node values of the latent, bottleneck layer of autoencoder(s) 105.

For example, a specific CDE 21 input (e.g., written text) may be received from a specific source 20 (e.g., an emailing application). An encoder 110 that is uniquely configured to encode CDEs 21 of the same type (e.g., encode textual email messages) may be trained to produce an invariant representation 110B of the CDE 21, in a latent vector space. As elaborated herein, encoder 110 may generate invariant representation 110B such that it retains significant information that pertains to a subject's condition (e.g., indicating a stroke), but omits information that pertains to format or origin of the input CDE 21 (e.g., a style of text).

According to some embodiments, system 100 may include one or more ML-based classification models 140, also denoted herein as “classifiers” 140 or “predictive models” 140.

As elaborated herein, the one or more predictive models 140 may be trained to receive at least one source-invariant representation 110B of at least one content data element 21, and produce a prediction data element 140A, based on the received source-invariant representation 110B. Prediction data element 140A may represent a predicted, or classified condition of the subject.

Pertaining to the example of a stroke condition, source-invariant representation 110B data element may include latent information, encoded from any one of sources 20 by respective, dedicated encoder(s) 110. This latent information may correspond to, or represent a condition of a stroke in a human subject. System 100 may include a predictive model 140 which may apply a ML-based function on the source-invariant representation 110B data element, to predict, or classify a condition of the relevant human subject, as one who is undergoing a stroke.

According to some embodiments, system 100 may include at least one adversarial neural network 130, configured to predict, based on the source-invariant representation 110B of an input CDE 21, an identification 130A of an origin data source or data platform 20, from which the input CDE 21 had originated.

For example, an input CDE 21 may be received from at least one source 20, as elaborated for example in relation to FIG. 2B. For example, input CDE 21 may originate from a textual data source 2100 such as an email 3130, an imaging source 2200, such as an image of the human subject 3240, and the like. As elaborated herein, the at least one adversarial neural network 130 may be trained to predict an identification of the source 20, from which input CDE 21 had been received. In this example, predicted identification 130A may include an alphanumeric label that may identify the origin source 20 (e.g., “Gmail”, “Tweeter”, “Mill scan”, etc.).

According to some embodiments, system 100 may include at least one first training module 150A, adapted to train the one or more autoencoders 105 against adversarial network 130 (which may attempt to identify the source of input CDE 21), given the invariant representation 110B of CDE 21.

In some embodiments, during an autoencoder training stage, training module 150A may receive a plurality of training content data elements 21 from a plurality of data sources 20. Training module 150A may train the one or more autoencoder modules 105, based on the plurality of training content data elements 21, to generate the source-invariant representation 110B such that one or more adversarial NNs 130 would fail in predicting the identification 130A of origin data sources 20 of one or more input CDEs 21 of the plurality of training CDEs.

In other words, training module 150A may train the encoder(s) 110 and decoder(s) 120 of one or more autoencoder modules 105 such that adversarial NN 130 may not be able to correctly produce a predicted identification 130A of an origin or source 20 of input CDE 21, based on invariant representation 110B.

According to some embodiments, the autoencoders restored version 120A of the input CDE 21 may include an identification of origin or source 20 of input CDE 21. Training module 150A may train encoder(s) 110 iteratively (e.g., repetitively, over a continuous process), using the output of respective decoder(s) 120 as supervisory data. In each iteration, the predicted identification 130A of source 20 may be compared to, or analyzed in relation to restored version 120A.

For example, if predicted identification 130A matches, or corresponds to an identification of origin or source 20 of input CDE 21, that is included in restored version 120A, then adversarial network 130 may be considered to have correctly predicted an origin or source 20 of input CDE 21, based on the current invariant representation 110B. In such a condition, training of autoencoder(s) 105 (e.g., of encoder(s) 110) may reiterate, to produce a new version of invariant representation data element 110B.

In a complementary example, if predicted identification 130A does not match an identification of origin or source 20 of input CDE 21, that is included in restored version 120A, then adversarial network 130 may be considered to have failed in predicting an origin or source 20 of input CDE 21. In such a condition, training of autoencoder(s) 105 (e.g., of encoder(s) 110) may halt, signifying that the present version of invariant representation data element 110B is sufficiently void of source-specific indications.

In some embodiments, the present invention may employ Adversarial Neural Networks (ANNs) and/or Generative Adversarial Networks (GANs). In some embodiments, an exemplary adversarial architecture of the present invention may provide for embedding a network in a latent space using autoencoder networks configured to generate a representation of an input in the latent space using an autoencoder model, and to generate a final representation of the input after the autoencoder model has been trained. A generative model is then configured to generate a representation of a set of samples in the latent space using a generative network that encodes as much information about the source data as possible. A discriminative network is then trained to discriminate between the representation of different data sources. A training module includes a processor configured to jointly train the autoencoders, the generative network, and the discriminative model.

Thus, in some embodiments, an adversarial architecture 130 of the present invention may include two neural networks which compete with each other, wherein a first network learns to map from a latent space to a data distribution of interest, while a second network attempts to distinguish candidates produced by the first network from the true data distribution. The first, generative, network's training objective is thus to increase the error rate of the second, discriminative, network by producing novel candidates that the discriminative network thinks are part of the true data distribution. The generative network thus trains based on whether it succeeds in fooling the discriminative network. In some embodiments the generative network is seeded with randomized input that is sampled from a predefined distribution (e.g., a multivariate normal distribution). Thereafter, candidates synthesized by the generative network are evaluated by the discriminative network. Independent backpropagation procedures are applied to both networks so that the generative network produces better candidates, while the discriminator becomes more skilled at identifying the data source.

In some embodiments, this architecture creates a competition between the autoencoders that are trying to preserve as much information about the source as possible, while minimizing the ability to recognize the data source. Hence, the result of such competition is a set of encoders that convert each data source into a representation that preserves most of the original data while making sure that the encoding from the different sources is indistinguishable. The autoencoders try to preserve the information while the adversarial part forces masking source-specific information.

In some embodiments, the generated invariant representations 110B from each of the available sources 20 may be grouped to form an invariant representations' dataset 110C. Dataset 110C may then be used for training and inference purposes of various machine learning prediction and classification models 140.

In some embodiments, the generated dataset 110C may be used to train one or more predictive ML models to predict various health-related states in users or human subjects. For example, the health-related states may be correlated with impaired and/or declining cognition in the users and/or may be manifested in particular cognitive symptoms.

Reference is further made to FIG. 4B, which is a schematic block diagram depicting aspects of system 100 for classifying or predicting a condition of a subject, according to some embodiments of the invention. It may be appreciated that system 100 depicted in FIG. 4B may be the same as system 100 depicted in FIG. 4A.

As shown in FIG. 4B, system 100 may include one or more second training modules 150B. One or more (e.g., each) training module 150B may correspond to one or more respective classification models 140.

According to some embodiments, during a classifier training stage, the one or more training modules 150B may be configured to receive a dataset 110C, which may include a plurality of source-invariant representations 110B corresponding to a respective plurality of training CDEs 21. Additionally, the one or more second training modules 150B may be configured to receive (e.g., from input 7 of FIG. 1) a plurality of annotation data elements 30, corresponding to the plurality of training CDEs 21.

For example, training CDEs 21 may include a plurality of data elements of different types or categories as elaborated herein (e.g., in relation to FIG. 2B), and annotation data elements 30 may be, or may include metadata such as labels or indications, provided by an expert (e.g., a human expert) regarding a condition of a relevant human subject. For example, a training CDEs 21 may include an image or a sequence of images (e.g., a video) of a human subject, and a corresponding annotation data elements 30 may include a label, or an indication, provided by an expert (e.g., a physician) regarding the subject's condition (e.g., whether the individual is undergoing a stroke).

According to some embodiments, during the classifier training stage, the one or more training modules 150B may train the one or more classification models 140 to produce one or more prediction data elements 140A, based on the plurality of source-invariant representation data elements 110B. In the process of training, the one or more training modules 150B may use the annotation data elements 30 as supervisory data, as known in the art.

In some embodiments, predictive models 140 trained based on the generated invariant data may be robust to changes in available data sources and usage trends, and therefore may be able to deliver accurate, source-independent prediction to a wider audience over a longer period of time. In some embodiments, the present invention provides for integrating privacy technology efficiently, thereby addressing a major regulatory and ethical challenge of such systems.

In some embodiments, a predictive machine learning model trained on a dataset 110C generated by encoding machine learning model(s) 110 may be invariant to permutations, wherein an output of the model does not change under any permutation of the elements in the input set of data sources. In some embodiments, encoding ML models 110 may be configured to process CDE 21 input sets of varying sizes.

In some embodiments, a predictive ML model trained on a dataset 110C generated by the encoder(s) 110 may benefit from aggregating training and inference data from a plurality of sources, and be invulnerable to changes in the number of aggregated sources and/or to the size of dataset 110C.

In some embodiments, a predictive ML model 140 trained on a dataset 110C generated by encoder ML model(s) 110 may be configured for supervised, unsupervised and/or semi-supervised training.

In some embodiments, a predictive ML model 140 trained on a dataset 110C generated by encoder ML model(s) 110 may provide for data privacy by being invariant, or oblivious to the sources of data.

In some embodiments, a predictive ML model 140 trained on a dataset 110C generated by encoder ML model(s) 110 may be configured for using encrypted data.

It may be appreciated by a person skilled in the art that operation of the one or more first training modules 150A (e.g., during the autoencoder 105 training stage) may compete with operation of the one or more second training modules 150B (e.g., during the predictive models' 140 training stage). For example, the one or more first training modules 150A may train ML based encoder(s) 110 such that invariant representation data elements 110B may be devoid of data that is indicative of a condition of a human subject, and may thus obstruct the function of the one or more predictive models 140.

In other words, the one or more first training modules 150A may collaborate with the one or more second training modules 150B, so as to avoid such obstruction.

For example, the one or more first training modules 150A configured to train the one or more autoencoders 105 may be the same entity as the one or more second training modules 150B configured to train the one or more predictive models 140. Alternatively, the one or more first training modules 150A may be communicatively connected to the one or more second training modules 150B.

In another example, the at least one first training module 150A may take the outcome 140A of one or more predictive models 140, during the autoencoder training stage. For example, the at least one first training module 150A may receive a plurality of annotation data elements 30, corresponding to the plurality of training CDEs 21, and receive, from the one or more classification models 140, a plurality of prediction data elements 140A, corresponding to the plurality of training content data elements. During the autoencoder training stage, the at least one first training module 150A may train the one or more autoencoder modules 105 further based on the prediction data elements 140A and annotation data elements 30.

Additionally, or alternatively, during the autoencoder stage, the at least one first training module 150A may conditionally train encoder 110 and/or decoder 120 and consequently change invariant representation 110B. For example, the at least one first training module 150A may train encoder 110 (e.g., change invariant representation 110B) only if the relevant classification model(s) 140 produce a correct prediction 140A of a condition of a human subject, based on the outcome representation data elements 110B. The at least one first training module 150A may ascertain that prediction 140A is “correct” if it matches, or corresponds to a label or indication included in annotation data element 30.

In other words, the plurality of annotation data elements 30 may represent ground-truth information pertaining to a condition of corresponding subjects. The at least one first training module 150A may be configured to train the one or more autoencoder modules 105 (e.g., encoder 100 and/or decoder 120) to generate the source-invariant representation 110B, such that the classification models 140 correctly predict 140A the conditions of relevant subjects, as represented by the annotation data elements 30.

Reference is now made also to FIG. 2B. According to some embodiments, the one or more encoder ML models 110 and the one or more adversarial networks 130 may be organized hierarchically, according to data source 20 types, as represented in the example of hierarchical categorization 200. In other words, encoder(s) 110 may include a plurality of ML-based encoder modules 110, that are arranged hierarchically, corresponding to data source 20 types.

For example, a top-level, or top hierarchy encoder 110 model and/or adversarial network module 130 may correspond to a general representation of CDE 21 source types (e.g., corresponding to generic representation sources 1000 of FIG. 2B). A second, subsequent hierarchy encoder 110 model and/or adversarial network module 130 may correspond to high-level source types (e.g., corresponding to textual/audio source types 2100, imaging source types 2200 and/or “omics” source types 2300 of FIG. 2B). A third, subsequent hierarchy encoder 110 model and/or adversarial network module 130 may correspond to higher granularity of data source subtypes (e.g., corresponding to subtype elements 3110, 3120, 3130, 3210, 3220, 3230, 3240, 3310 and 3320 of FIG. 2B). A fourth subsequent hierarchy encoder 110 model and/or adversarial network module 130 may correspond to yet higher granularity, and may represent specific platforms and source types (e.g., corresponding to data source elements 4111, 4112, 4113 of FIG. 2B). Additional hierarchical levels may also be possible.

In such embodiments, invariant representation 110B may be or may include a data structure (e.g., a vector, an array, and the like) which may include separate entries from each of the hierarchy-specific encoders 110. For example, invariant representation 110B may include a first group of entries, representing a feature vector in a latent space of a first encoder 110 corresponding to a first hierarchical level (e.g., textual/audio source types 2100, in level B of FIG. 2B), and a second group of entries, representing a feature vector in a latent space of a second encoder 110 corresponding to a second hierarchical level (e.g., social networks 3110, in level C of FIG. 2B).

In other words, applying the one or more autoencoder models on at least one CDE 21 may generate a feature vector 110A that includes a plurality of source-invariant representations 110B of the CDE 21, where each source-invariant representation 110B of the feature vector 110A corresponds to a respective hierarchical level.

For example, a textual CDE 21 may be obtained from a “WhatsApp” message. Corresponding invariant representations 110B may include a first entry from an encoder 10 corresponding to level D (e.g., 4113), a second entry from an encoder 110 corresponding to level C (e.g., 3110) and a third entry from an encoder 110 corresponding to level B (e.g., 2100).

Additionally, or alternatively, encoders 110 may include one or more encoders elements that are common to a plurality of hierarchical levels of hierarchical categorization 200. As elaborated herein, in such embodiments, encoders 110 may be trained according to a penalization or weighing system, to produce an invariant representation 110B that would pertain to a highest hierarchical level (e.g., be common to as many CDE 21 sources or origins) as possible. In such embodiments, invariant representation 110B may be or may include a data structure (e.g., a vector, an array, and the like) that represents a feature vector 110A in a latent space of an encoder 110 that represents a common denominator of a plurality of CDE 21 sources.

For example, a textual CDE 21 may be obtained from a “WhatsApp” message. A corresponding invariant representation 110B may include an entry from an encoder 110 trained to represent CDE 21 in the highest level possible.

In other words:
(A) If CDE 21 may be represented such that invariant representation 110B would enable predictive models 140 to correctly predict a condition of the subject, while adversary network 130 would fail to distinguish the origin of CDE 21 between a WhatsApp textual message and an MRI scan, then invariant representation 110B would pertain to level A (e.g., the common denominator for imaging sources and textual sources);
(B) If, on the other hand, CDE 21 may be represented such that invariant representation 110B would enable predictive models 140 to correctly predict the condition of the subject, while adversary network 130 would fail to distinguish the origin of CDE 21 between a WhatsApp textual message and an email, then invariant representation 110B would pertain to level B (e.g., the common denominator for textual sources);
(C) If, CDE 21 may be represented such that invariant representation 110B would enable predictive models 140 to correctly predict the condition of the subject, while adversary network 130 would fail to distinguish the origin of CDE 21 between a WhatsApp textual message and a Facebook post, then invariant representation 110B would pertain to level C (e.g., the common denominator between WhatsApp texts and Facebook posts), etc.

According to some embodiments, during an autoencoder training stage, training module 150A may train the one or more encoders 110 such that invariant representation 110B may correspond to the highest possible hierarchical level of hierarchical categorization 200.

For example, during an autoencoder training stage, training module 150A may penalize usage of a low-level encoder 110 according to the encoder's 110 level in hierarchical categorization 200. In other words, high penalty or cost may be assigned to an encoder 110 in a low level (e.g., level D of FIG. 2B) of hierarchical categorization 200, and low penalty or cost may be assigned to a high level (e.g., levels A or B of FIG. 2B) of hierarchical categorization 200.

Additionally, or alternatively, during the autoencoder training stage training module 150A may receive a plurality of CDEs 21 (denoted herein as training CDEs 21) from a plurality of data sources 20. Training module 150A may train the one or more autoencoder modules 110, based on the plurality of training content data elements, to generate said feature vector, while applying a predetermined weight to each source-invariant representations of the feature vector. The predetermined weight value may be a numerical value (e.g., in the range of [0-10]). The predetermined weight value may be determined according to the hierarchical level of the respective source-invariant representation.

According to some embodiments, for each pair of source-invariant representations 110B, that includes a first source-invariant representation 110B corresponding to a first hierarchical level (e.g., C), and a second source-invariant representation 110B corresponding to a second, higher hierarchical level (e.g., B), the weight of the second source-invariant representation may be predetermined as higher than the weight of the first source-invariant representation.

It may be appreciated that such weighing or penalizing may drive the training of the one or more encoder modules 110 toward a high-level hierarchical representation.

Pertaining to the example of the CDE 21 of the WhatsApp text message, it may be appreciated that applying such weights may drive the trained encoders 110 more towards the higher levels, such as level A (e.g., being oblivious to difference between imaging sources 20 and textual sources) and less toward the lower levels, such as level D (e.g., being able to differentiate only between WhatsApp messages and Facebook posts).

Additionally, during the autoencoder training stage, adversarial network 130 may only be allowed to attempt to produce a prediction 130A of the identification of an origin source of a CDE 21, by using the sections that are common to the two sources that it is trying to distinguish between.

For example, if adversarial network 130 is trying to tell whether CDE 21 originated from an MRI image or an Xray image, it may only be allowed to use an invariant representation data element 110B obtained by an encoder 110 that corresponds to imaging sources 2200 of hierarchical categorization 200. It may not be allowed to use invariant representation data element 110B obtained by an encoder 110 that corresponds to MM-specific data sources 3220 or X-ray-specific data sources 3210. Thus, embodiments of the invention may “push” system 100 to use encoder(s) 110 which use the more generic sections as much as possible (e.g., higher levels of hierarchical categorization 200), but may still not avoid using information that is unique to a certain modality.

In a similar manner, classification models 140 may also be trained to use data at specific sections and prefer the more generic sections as much as possible.

For example, as elaborated herein, during the classifier training stage, training module 150B may receive a plurality of feature vectors 110A, corresponding to a respective plurality of CDEs 21 (denoted herein as training CDEs 21). Training module 150B may also receive a respective plurality of annotation data elements 30, corresponding to the plurality of training content data elements, and may train the one or more classification models 140 (also referred to herein as predictive models 140), to produce the prediction data elements 140A, based on the plurality of source-invariant representations 110B of feature vectors 110A, while using the annotation data elements as supervisory data. Additionally, training module 150B may applying a predetermined training weight to each source-invariant representations 110B of feature vector 110A. This training weight may be determined according to the hierarchical level of the respective source-invariant representation.

In other words, during a predictive model (or classifier) training stage, classification models 140 may be penalized against using invariant representation data elements 110B obtained from encoders corresponding to low-level sections (e.g., level D of FIG. 2B), so as to prefer representation data elements 110B obtained from encoders corresponding to higher level sections (e.g., levels A, B of FIG. 2B). It may be appreciated that such training may produce models 140 that would be as generic, and source-invariant as possible.

Reference is now made to FIG. 4C which is a schematic block diagram depicting additional or alternative aspects of system 100 for classifying or predicting a condition of a subject, according to some embodiments of the invention. It may be appreciated that system 100 depicted in FIG. 4C may be the same as system 100 depicted in FIG. 4A.

As shown in FIG. 4C, following training of ML-based encoders 110 and/or ML-based predictive models (or classifiers) 140, system 100 may be used to infer these ML-based models so as predict a mental or cognitive condition of a human subject, based on historical and/or new input of CDEs 21.

In other words, during an inference stage, encoders 110 may be used to produce a source-invariant representation 110B of incoming CDEs 21, and predictive models 140 may be used to classify, or predict 140A a physical, mental and/or cognitive state or condition of a human subject, associated with CDEs 21, based on the source-invariant representations 110B.

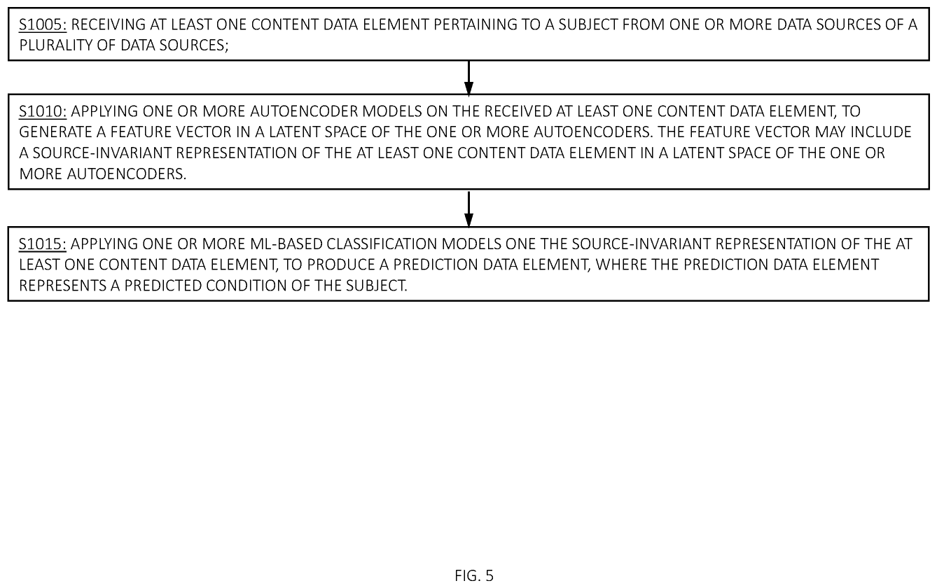

Reference is now made to FIG. 5 which is a schematic flow diagram depicting a method of classifying or predicting, by at least one processor, a condition (e.g., a medical, mental and/or cognitive condition) of a subject, based on invariant representation 110B of content data elements 21, according to some embodiments of the invention.

As shown in step S1005, the at least one processor (e.g., processor 2 of FIG. 1) may be configured to receiving at least one content data element 21 pertaining to the subject from one or more data sources 20 of a plurality of data sources 20.

As shown in step S1010, the at least one processor 2 may apply one or more autoencoder models (e.g., A.E. 105 of FIG. 4A) on the received at least one content data element 21, to generate a feature vector (e.g., element 110A of FIG. 4A) in a latent space of the one or more autoencoders 105. The feature vector may include a source-invariant representation 110B of the at least one content data element 21.

As shown in step S1015, the at least one processor 2 may apply one or more ML-based classification models (e.g., predictive models 140 of FIG. 4A) one the source-invariant representation 110B of the at least one content data element 21, to produce a prediction data element 140A. The prediction data element 140A may represent a predicted condition (e.g., a medical, mental, and/or cognitive condition) of the subject.

Embodiments of the invention include a practical application for assessing a condition such as a medical condition and or a mental or cognitive condition of a subject.

Embodiments of the invention may provide several improvements in assistive diagnosis technology.

For example, a physician may be required to assess a mental condition of a subject (e.g., a patient), who may be suspected as suffering from a mental or cognitive degenerative disease or condition. In such situations, the physician may be limited to produce their assessment based on limited, concurrent information such as results of physical examination, or content of relevant medical records, if exist.

As elaborated herein, embodiments of the invention may utilize existing information (e.g., online, internet-based information) referred to herein as CDEs 21, which may be accumulated over time by the subject (e.g., following the patient's online actions) and/or in relation to the subject (e.g., by other people interacting with the patient). It may be appreciated that such accumulation of data over time may reflect data that may not be available to the physician.

For example, a condition in which a patient may draft textual messages (e.g., on social networking platforms 3110) which change over time (e.g., having an increasingly large numbers of spelling mistakes, or using a different style) may predict a process of cognitive deterioration (e.g., Alzheimer's disease), an onset of medical condition (e.g., a cerebral stroke), a development of a mental state (e.g., acute depression), and the like.

Additionally, as elaborated herein, embodiments of the invention may effectively gather and integrate information that pertains to many sources, types and platforms for the purpose of classification of the patient's condition. This is done as elaborated herein by training autoencoders 105 (e.g., and subsequently encoders 110) against adversarial networks 130, such that characteristics or properties that are unique to specific data sources 20 may become undetectable by adversarial networks 130, thus allowing integration of the different types of data for subject condition classification.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system for predicting a condition of a subject, the system comprising:
one or more autoencoder modules, trained to:
receive at least one content data element pertaining to the subject from one or more data sources of a plurality of data sources; and
generate a feature vector in a latent space of the one or more autoencoders, said feature vector comprising a source-invariant representation of said at least one content data element, and
one or more machine-learning (ML) based classification models, trained to:
receive the source-invariant representation of the at least one content data element; and
produce a prediction data element, representing a predicted condition of the subject, based on the source-invariant representation of said at least one content data element.

2. The system of claim 1, further comprising at least one adversarial neural network (NN) configured to predict, based on the source-invariant representation of the at least one content data element, an identification of an origin data source from which the at least one content data element originated.

3. The system of claim 2, further comprising at least one first training module configured to, during an autoencoder training stage:
receive a plurality of training content data elements from a plurality of data sources; and
train the one or more autoencoder modules, based on the plurality of training content data elements, to generate the source-invariant representation such that the adversarial NN would fail in predicting the identification of origin data sources of one or more content data elements of the plurality of training content data elements.

4. The system of claim 3, wherein the at least one first training module is further configured to, during the autoencoder training stage:
receive a plurality of annotation data elements, corresponding to the plurality of training content data elements;
receive, from the one or more classification models, a plurality of prediction data elements, corresponding to the plurality of training content data elements; and
train the one or more autoencoder modules further based on the prediction data elements and annotation data elements.

5. The system of claim 4, wherein the plurality of annotation data elements represent ground-truth information pertaining to a condition of corresponding subjects, and wherein the at least one first training module is configured to train the one or more autoencoder modules to generate the source-invariant representation, such that the classification models correctly predict the conditions of relevant subjects, as represented by the annotation data elements.

6. The system of claim 1, further comprising one or more second training modules, corresponding to the respective one or more classification models, wherein the one or more second training modules are configured to, during a classifier training stage:
receive a plurality of source-invariant representations of a respective plurality of training content data elements;
receive a plurality of annotation data elements, corresponding to the plurality of training content data elements; and
train the one or more classification models to produce the prediction data elements, based on the plurality of source-invariant representations, using the annotation data elements as supervisory data.

7. A method of predicting a condition of a subject by at least one processor, the method comprising:
receiving at least one content data element pertaining to the subject from one or more data sources of a plurality of data sources;
applying one or more autoencoder models on the received at least one content data element, to generate a feature vector in a latent space of the one or more autoencoders, said feature vector comprising a source-invariant representation of said at least one content data element; and
applying one or more ML-based classification models one the source-invariant representation of the at least one content data element, to produce a prediction data element,
wherein said prediction data element represents a predicted condition of the subject.

8. The method of claim 7, further comprising, during an autoencoder training stage:
receiving a plurality of training content data elements from a plurality of data sources;
applying at least one adversarial NN on the source-invariant representation of the at least one content data element, to produce an identification of an origin data source, from which the at least one content data element was received; and
training the one or more autoencoder modules, based on the plurality of training content data elements, to generate the source-invariant representation such that the at least one adversarial NN would fail in predicting the identification of origin data sources of one or more content data elements of the plurality of training content data elements.

9. The method of claim 8, further comprising, during the autoencoder training stage:
receiving a plurality of annotation data elements, corresponding to the plurality of training content data elements;
receiving, from the one or more classification models, a plurality of prediction data elements, corresponding to the plurality of training content data elements; and
training the one or more autoencoder modules, further based on the prediction data elements and annotation data elements.

10. The method according to claim 9, wherein the plurality of annotation data elements represent ground-truth information pertaining to a condition of corresponding subjects, and wherein the method further comprises training the one or more autoencoder modules to generate the source-invariant representation, such that the classification models correctly predict the conditions of relevant subjects, as represented by the annotation data elements.

11. The method of claim 7, further comprising, during a classifier training stage:
receiving a plurality of source-invariant representations of a respective plurality of training content data elements;
receiving a plurality of annotation data elements, corresponding to the plurality of training content data elements; and
training the one or more classification models to produce the prediction data elements, based on the plurality of source-invariant representations, using the annotation data elements as supervisory data.

12. The method of claim 7, further comprising receiving a definition of a hierarchical categorization data structure, representing a plurality of hierarchical levels of the received content data elements,
wherein applying the one or more autoencoder models on at least one content data element comprises generating a feature vector that comprises a plurality of source-invariant representations of said at least one content data element,
and wherein each source-invariant representation of the feature vector corresponds to a respective hierarchical level.

13. The method of claim 12, further comprising, during an autoencoder training stage:
receiving a plurality of training content data elements from a plurality of data sources; and
training the one or more autoencoder modules, based on the plurality of training content data elements, to generate said feature vector, while applying a predetermined weight to each source-invariant representations of the feature vector,
wherein said weight is determined according to the hierarchical level of the respective source-invariant representation.

14. The method of claim 13, wherein for each pair of source-invariant representations, said pair comprising a first source-invariant representation corresponding to a first hierarchical level, and a second source-invariant representation corresponding to a second, higher hierarchical level, the weight of the second source-invariant representation is higher than the weight of the first source-invariant representation.

15. The method of claim 12, further comprising, during a classifier training stage:
receiving a plurality of feature vectors, corresponding to a respective plurality of training content data elements;
receiving a plurality of annotation data elements, corresponding to the plurality of training content data elements; and
training the one or more classification models to produce the prediction data elements, based on the plurality of source-invariant representations, while (a) using the annotation data elements as supervisory data, and (b) applying a predetermined weight to each source-invariant representations of the feature vector,
wherein said weight is determined according to the hierarchical level of the respective source-invariant representation.

16. The method of claim 7, wherein the at least one content data element is selected from a list of textual or audible data sources, consisting of: an Internet search query, a posting to a social network by the subject, an email pertaining to the subject, a text message pertaining to the subject, a transcription of a voice command pertaining to the subject, and text included in a medical record pertaining to the subject.

17. The method of claim 7, wherein the at least one content data element is selected from a list of online data sources, consisting of: online user-selections performed by the subject, online images pertaining to the subject, online videos pertaining to the subject, and online audio or vocal data elements pertaining to the subject.

18. The method of claim 7, wherein the at least one content data element is selected from a list of image data sources, consisting of: an image of the subject, a video of the subject, a Magnetic Resonance Imaging (MM) scan of the subject, a Computed Tomography (CT) scan of the subject, and images obtained from an Ultrasound (US) scan of the subject.

19. The method of claim 7, wherein the at least one content data element is selected from a list consisting of a proteomic data element and a genomic data element.

20. A system for predicting a condition of a subject, the system comprising: a non-transitory memory device, wherein modules of instruction code are stored, and at least one processor associated with the memory device, and configured to execute the modules of instruction code, whereupon execution of said modules of instruction code, the at least one processor is configured to:
receive at least one content data element pertaining to the subject from one or more data sources of a plurality of data sources;
apply one or more autoencoder modules on the at least one content data element to generate a feature vector in a latent space of the one or more autoencoders, said feature vector comprising a source-invariant representation of said at least one content data element, and
apply one or more machine-learning (ML) based classification models on the source-invariant representation of the at least one content data element, to produce a prediction data element, representing a predicted condition of the subject.
